(19) United States
(12) Patent Application Publication
(10) Pub. No.: US 2016/0122264 A1
(43) Pub. Date: May 5, 2016

(54) METHOD FOR THE OXIDATIVE DEHYDRATION OF N-BUTENES INTO 1,3-BUTADIEN

(71) Applicant: BASF SE, Ludwigshafen (DE)

(72) Inventors: GERHARD OLBERT, Dossenheim (DE); Gauthier Luc Maurice AVERLANT, Darmstadt (DE); Philip GRÜNE, Mannheim (DE); Jan Pablo JOSCH, Neustadt (DE)

(73) Assignee: BASF SE, Ludwigshafen (DE)

(21) Appl. No.: 14/898,849

(22) PCT Filed: Jun. 16, 2014
(86) PCT No.: PCT/EP2014/062505

§ 371 Date: Dec. 16, 2015

(30) Foreign Application Priority Data
Jun. 17, 2013 (EP) 13172318.1

(51) Int. Cl. C07C 5/48; B01J 8/06

(52) U.S. Cl. CPC C07C 5/48; B01J 8/067; B01J 2208/00212; B01J 2208/00176; B01J 2208/065

(57) ABSTRACT

The invention relates to a method for producing 1,3 butadien by means of the oxidative dehydration of n-butenes on a heterogenous particulate multimetal oxide catalyst which contains molybdenum as the active compound and at least one other metal and which is filled into the contact tubes (KR) of two or more tube bundle reactors (R-I, R-II), wherein a heat transfer medium flows around the intermediate space between the contact tubes (KR) of the two or more tube bundle reactors (R-I, R-II). The method includes a production mode and a regeneration mode which are carried out in an alternating manner. In the production mode, an n-butene-containing feed flow is mixed with an oxygen-containing gas flow and conducted as a supply flow (1) over the heterogenous particulate multimetal oxide catalyst filled into the contact tubes (KR) of the two or more tube bundle reactors (R-I, R-II), and the heat transfer medium absorbs the released reaction heat, minus the heat quantity used to heat the supply flow (1) to the reaction temperature in the production mode, by means of an indirect heat exchange and completely or partly dispenses the reaction heat onto a secondary heat transfer medium (H2Oliq) in an external cooler (SBK). In the regeneration mode, the heterogenous particulate multimetal oxide catalyst is regenerated by conducting an oxygen-containing gas mixture (3) over the catalyst and burning off the deposits accumulated on the heterogenous particulate multimetal oxide catalyst. The invention is characterized in that the two or more tube bundle reactors (R-I, R-II) have a single heat transfer medium circuit and as many of the two or more tube bundle reactors (R-I, R-II) as necessary are operated constantly in the production mode so that the released reaction heat, minus the heat quantity used to heat the supply flow (1) to the reaction temperature in the production mode, suffices to keep the temperature of the heat transfer medium in the intermediate spaces between the content tubes (KR) of all the tube bundle reactors (R-I, R-II) at a constant level with a variation range of maximally +/−10 DEG C.

### CROSS-REFERENCE TO RELATED APPLICATIONS

This application is a national stage application (under 35 U.S.C. §371) of PCT/EP2014/062505, filed Jun. 16, 2014, which claims benefit of European Application No. 13172318.1, filed Jun. 17, 2013, both of which are incorporated herein by reference in their entirety.

### DESCRIPTION

The invention relates to a process for the oxidative dehydrogenation of n-butenes to 1,3-butadiene.

1,3-Butadiene is an important basic chemical and is used, for example, for the preparation of synthetic rubbers (1,3-butadiene hompolymers, styrene-1,3-butadiene rubber or nitrile rubber) or for the preparation of thermoplastic terpolymers (acrylonitrile-1,3-butadiene-styrene copolymers). 1,3-Butadiene is also converted into sulfolane, chloroprene and 1,4-hexamethylenediamine (via 1,4-dichlorobutene and adiponitrile). Furthermore, 1,3-butadiene can be dimerized to produce vinylcyclohexene which can be dehydrogenated to form styrene.

1,3-Butadiene can be prepared by thermal cracking (steam cracking) saturated hydrocarbons, with naphtha usually being used as raw material. The steam cracking of naphtha gives a hydrocarbon mixture of methane, ethane, ethene, acetylene, propane, propene, propyne, allene, butanes, butenes, 1,3-butadiene, butynes, methylallene, C₅-hydrocarbons and higher hydrocarbons.

1,3-Butadiene can also be obtained by oxidative dehydrogenation of n-butenes (1-butene and/or 2-butene). Any mixture comprising n-butenes can be used as feedstream mixture for the oxidative dehydrogenation of n-butenes to 1,3-butadiene. For example, it is possible to use a fraction which comprises n-butenes (1-butene and/or 2-butene) as main constituent and has been obtained from the C₄ fraction from a naphtha cracker by removal of 1,3-butadiene and isobutene. Furthermore, gas mixtures which comprise 1-butene, cis-2-butene, trans-2-butene or mixtures thereof and have been obtained by dimerization of ethylene can also be used as feedstream. In addition, gas mixtures which comprise n-butenes and have been obtained by fluid catalytic cracking (FCC) can be used as feedstream.

Gas mixtures which comprise n-butenes and are used as feedstream in the oxidative dehydrogenation of n-butenes to 1,3-butadiene can also be prepared by nonoxidative dehydrogenation of gas mixtures comprising n-butane.

WO 2009/124945 discloses a coated catalyst for the oxidative dehydrogenation of 1-butene and/or 2-butene to 1,3-butadiene, which can be obtained from a catalyst precursor comprising
(a) a support body and
(b) a shell comprising a catalytically active multimetal oxide which comprises molybdenum and at least one further metal and has the general formula

Mo₁₂BiₐCr_{b}X¹_{c}Fe_{d}X²ₑX³_{f}O_{y}
where
X¹=Co and/or Ni,
X²=Si and/or Al,
X³=Li, Na, K, Cs and/or Rb,
0.2≦a≦1
0≦b≦2,
2≦c≦10,
0.5≦d≦10,
0≦e≦10,
0≦f≦0.5, and
y=a number which is determined by the valence and abundance of the elements other than oxygen in order to achieve charge neutrality,
and at least one pore former.

WO 2010/137595 discloses a multimetal oxide catalyst for the oxidative dehydrogenation of alkenes to dienes, which comprises at least molybdenum, bismuth and cobalt and has the general formula

MoₐBi_{b}Co_{c}Ni_{d}FeₑX_{f}Y_{g}ZₕSiᵢOⱼ

In this formula, X is at least one element selected from the group consisting of magnesium (Mg), calcium (Ca), zinc (Zn), cerium (Ce) and samarium (Sm). Y is at least one element selected from the group consisting of sodium (Na), potassium (K), rubidium (Rb), cesium (Cs) and thallium (TI). Z is at least one element selected from the group consisting of boron (B), phosphorus (P), arsenic (As) and tungsten (W). a-j are the atom fractions of the respective element, where a=12, b=0.5-7, c=0-10, d=0-10, (where c+d=1-10), e=0.05-3, f=0-2, g=0.04-2, h=0-3 and i=5-48. In the examples, a catalyst having the composition Mo₁₂Bi₅Co_{2.5}Ni_{2.5}Fe_{0.4}Na_{0.35}B_{0.2}K_{0.08}Si₂₄ in the form of pellets having a diameter of 5 mm and a height of 4 mm is used in the oxidative dehydrogenation of n-butenes to 1,3-butadiene.

In the oxidative dehydrogenation of n-butenes to 1,3-butadiene, precursors of carboneous material, for example styrene, anthraquinone and fluorenone, which can ultimately lead to carbonization and deactivation of the multimetal oxide catalyst can be formed. The formation of carbon-comprising deposits can increase the pressure drop over the catalyst bed. It is possible to burn off the carbon-comprising deposits on the multimetal oxide catalyst at regular intervals by means of an oxygen-comprising gas in order to regenerate the catalyst and restore the activity of the catalyst.

JP 60-058928 describes the regeneration of a multimetal oxide catalyst for the oxidative dehydrogenation of n-butenes to 1,3-butadiene, which comprises at least molybdenum, bismuth, iron, cobalt and antimony, by means of an oxygen-comprising gas mixture at a temperature of from 300 to 700° C., preferably from 350 to 650° C., and an oxygen concentration of from 0.1 to 5% by volume. Air which is diluted with suitable inert gases such as nitrogen, steam or carbon dioxide is introduced as oxygen-comprising gas mixture.

WO 2005/047226 describes the regeneration of a multimetal oxide catalyst for the partial oxidation of acrolein to acrylic acid, which comprises at least molybdenum and vanadium, by passing an oxygen-comprising gas mixture over the catalyst at a temperature of from 200 to 450° C. Lean air comprising from 3 to 10% by volume of oxygen is preferably used as oxygen-comprising gas mixture. Apart from oxygen and nitrogen, the gas mixture can comprise steam.

In the light of the above, it was an object of the invention to provide a process for the oxidative dehydrogenation of n-butenes to 1,3-butadiene, in which the regeneration of the multimetal oxide catalyst is very effective and simple.

The object is achieved by a process for preparing 1,3-butadiene by oxidative dehydrogenation of n-butenes over a heterogeneous particulate multimetal oxide catalyst which comprises molybdenum and at least one further metal as active composition and has been introduced into the catalyst tubes of two or more shell-and-tube reactors, where a heat transfer medium flows through the intermediate space between the catalyst tubes of the two or more shell-and-tube reactors, and the process comprises a production mode and a regeneration mode which are operated alternately,

in the production mode, a feedstream comprising the n-butenes is mixed with an oxygen-comprising gas stream and passed over the heterogeneous particulate multimetal oxide catalyst which has been introduced into the catalyst tubes of the two or more shell-and-tube reactors and the heat transfer medium takes up, by indirect heat exchange, the heat of reaction liberated minus the quantity of heat which is consumed for heating the input stream to the reaction temperature in the production mode and passes all or part of it onto a secondary heat transfer medium in an external cooler and, in the regeneration mode, the heterogeneous particulate multimetal oxide catalyst is regenerated by passing an oxygen-comprising gas mixture over the catalyst and burning off the deposits which have deposited on the heterogeneous particulate multimetal oxide catalyst, wherein
the two or more shell-and-tube reactors have a single heat transfer medium circuit and
the number of the two or more shell-and-tube reactors which are operated in the production mode is always such that the heat of reaction liberated minus the quantity of heat consumed for heating the input stream to the reaction temperature in the production mode is sufficient to keep the temperature of the heat transfer medium in the intermediate spaces between the catalyst tubes of all shell-and-tube reactors constant with a fluctuation range of not more than +/−10° C.

### A BRIEF DESCRIPTION OF THE FIGURES

FIG. 1 illustrates a process layout according to the prior art (1-reactor design);

FIG. 2 illustrates a preferred process layout according to the invention (2-reactor design), where FIG. 2 depicts only the plant components relevant for conveying the gas streams both in the production mode and in the regeneration mode;

FIGS. 3A, 3B, 3C schematic depictions of a preferred process layout according to the invention (2-reactor design), where the plant components relevant for conveying the heat transfer medium are depicted;

FIG. 4 illustrates a cross-sectional view through a particularly preferred, compact embodiment of a plant according to the invention (twin reactor);

FIG. 5 is along the section A-A from FIG. 4;

FIG. 6 is along the section B-B from FIG. 4;

FIGS. 7A, 7B illustrate cross-sectional views through deflection plates DS which extend over the cross section of the two reactors and the intermediate chamber Z and leave passages free in the outer regions facing away from one another of the two reactors R-I. R-II or are configured as two disk-shaped deflection plates RS.

It has been found that regeneration of the rnultimetal oxide catalyst can be carried out in a simple way at the elevated temperatures of at least 350° C. which are essential for achieving activity and selectivity of the catalyst without the use of external heaters, in particular electric heaters or combustion chambers, being necessary for this purpose, except for start-up of the plant. As a result of the way of carrying out the process according to the invention, it is also not necessary to reheat the reactor to the reaction temperature, in particular about 380° C., for the production mode after the regeneration, for which there has hitherto not been a reliable technical solution: electric heaters as have been used hitherto are not suitable for the frequent change in operating mode of large-scale reactors; they tend, in particular because of the high proportion of ceramic materials, to suffer from damage and malfunctions and are also expensive to operate.

In particular, the way of carrying out the process according to the invention also ensures a continuous input stream for downstream process stages, with load fluctuations in a range of not more than about 50 to 120% relative to the nominal capacity.

### Oxidative Dehydrogenation (Oxydehydrogenation, ODH) (Production Mode)

The production mode of the oxidative dehydrogenation of n-butenes to 1,3-butadiene is carried out by mixing a feedstream comprising n-butenes with an oxygen-comprising gas stream and optionally additional inert gas or steam and passing it over the heterogeneous particulate multimetal oxide catalyst which has been introduced into the catalyst tubes of two or more shell-and-tube reactors at a temperature of from 330 to 490° C. The temperatures mentioned relate to the temperature of the heat transfer medium.

The reaction temperature of the oxydehydrogenation is generally controlled by means of a heat transfer medium which circulates in the intermediate space around the catalyst tubes. Possible liquid heat transfer media of this type are, for example, melts of salts such as potassium nitrate, potassium nitrite, sodium nitrite and/or sodium nitrate and also melts of metals such as sodium, mercury and alloys of various metals. However, ionic liquids or heat transfer oils can also be used. The temperature of the heat transfer medium is from 330 to 490° C., preferably from 350 to 450° C. and particularly preferably from 365 to 420° C.

The above temperatures of the heat transfer medium are set by the heat transfer medium taking up the heat of reaction liberated in the oxydehydrogenation minus the quantity of heat which is consumed for heating the input stream to the reaction temperature in the production mode and passing this on partly or completely to a secondary heat transfer medium in an external cooler, which in a preferred embodiment is configured as a salt bath cooler. The secondary heat transfer medium can advantageously be water which can be utilized in the external cooler for generating steam.

In the feed lines to the external cooler for the heat transfer medium which flows around the catalyst tubes, there are regulable shutoff devices, in a preferred embodiment salt bath slide valves, via which the flow of the heat transfer medium can be regulated.

Owing to the exothermic nature of the reactions which occur, the temperature in particular sections in the interior of the catalyst tubes during the reaction can be higher than that of the heat transfer medium and hot spots are formed. The position and magnitude of the hot spot is determined by the reaction conditions, but can also be regulated via the dilution ratio of the catalyst bed or the flow of mixed gas.

The oxydehydrogenation is carried out in the catalyst tubes of two or more shell-and-tube reactors.

The heterogeneous particulate multimetal oxide catalyst comprises molybdenum and at least one further metal as active composition.

Multimetal oxide catalysts suitable for oxydehydrogenation are generally based on an Mo—Bi—O-comprising multimetal oxide system which generally additionally comprises iron. In general, the catalyst system comprises further additional components from groups 1 to 15 of the Periodic Table, for example potassium, cesium, magnesium, zirconium, chromium, nickel, cobalt, cadmium, tin, lead, germanium, lanthanum, manganese, tungsten, phosphorus, cerium, aluminum or silicon. Iron-comprising ferrites have also been proposed as catalysts.

In a preferred embodiment, the multimetal oxide comprises cobalt and/or nickel in addition to molybdenum. In a further preferred embodiment, the multimetal oxide comprises chromium. In a further preferred embodiment, the multimetal oxide comprises manganese.

In general, the catalytically active multimetal oxide comprising molybdenum and at least one further metal has the general formula (I),

Mo₁₂BiₐFe_{b}Co_{c}Ni_{d}CrₑX¹_{f}X²_{g}Oₓ (I)

where the variables have the following meanings:
X¹=W, Sn, Mn, La, Ce, Ge, Ti, Zr, Hf, Nb, P, Si, Sb, Al, Cd and/or Mg;
X²=Li, Na, K, Cs and/or Rb,
a=0.1 to 7, preferably from 0.3 to 1.5;
b=0 to 5, preferably from 2 to 4;
c=0 to 10, preferably from 3 to 10;
d=0 to 10;
e=0 to 5, preferably from 0.1 to 2;
f=0 to 24, preferably from 0.1 to 2;
g=0 to 2, preferably from 0.01 to 1; and
x=a number which is determined by the valence and abundance of the elements other than oxygen in (I).

The catalyst used according to the invention can be an all-active catalyst or a coated catalyst. If it is a coated catalyst, it has a support which is enveloped by a shell comprising the above-described active composition.

Support materials suitable for coated catalysts are, for example, porous or preferably nonporous aluminum oxides, silicon dioxide, zirconium dioxide, silicon carbide or silicates such as magnesium silicate or aluminum silicate (e.g. steatite of the type C 220 from CeramTec). The materials of the support bodies are chemically inert. The support material is preferably nonporous (ratio of the total volume of the pores to the volume of the support body preferably 1%).

The use of essentially nonporous, spherical supports composed of steatite (e.g. steatite of the type C 220 from CeramTec) and having a rough surface and a diameter of from 1 to 8 mm, preferably from 2 to 6 mm, particularly preferably from 2 to 3 or from 4 to 5 mm, is particularly useful. However, the use of cylinders composed of chemically inert support material as support bodies, whose length is from 2 to 10 mm and whose external diameter is from 4 to 10 mm, is also useful. In the case of rings as support bodies, the wall thickness is also usually from 1 to 4 mm. Preferred ring-shaped support bodies have a length of from 2 to 6 mm, and an external diameter of from 4 to 8 mm and a wall thickness of from 1 to 2 mm. Rings having the geometry 7 mm×3 mm×4 mm (external diameter×length×internal diameter) are particularly useful as support bodies. The layer thickness of the shell of a multimetal oxide composition comprising molybdenum and at least one further metal is generally from 5 to 1000 μm. Preference is given to from 10 to 800 μm, particularly preferably from 50 to 600 μm and very particularly preferably from 80 to 500 μm.

Examples of Mo—Bi—Fe—O-comprising multimetal oxides are Mo—Bi—Fe—Cr—O— or Mo—Bi—Fe—Zr—O-comprising multimetal oxides. Preferred systems are described, for example, in U.S. Pat. No. 4,547,615 (Mo₁₂BiFe_{0.1}Ni₈ZrCr₃K_{0.2}Oₓ and Mo₁₂BiFe_{0.1}Ni₈AlCr₃K_{0.2}Oₓ), U.S. Pat. No. 4,424,141 (Mo₁₂BiFe₃Co_{4.5}Ni_{2.5}P_{0.5}K0.1Oₓ+SiO₂), DE-A 25 30 959 (Mo₁₂BiFe₃Co_{4.5}Ni_{2.5}Cr0.5K0.1Oₓ, Mo13.75BiFe₃Co4.5Ni_{2.5}Ge0.5K0.8Oₓ, Mo₁₂BiFe₃Co_{4.5}Ni_{2.5}Mn_{0.5}K_{0.1}Oₓ and Mo₁₂BiFe₃Co4.5Ni2.5La0.5K_{0.1}Oₓ), U.S. Pat. No. 3,911,039 (Mo₁₂BiFe₃Co_{4.5}Ni_{2.5}Sn0.5K_{0.1}Oₓ), DE-A 25 30 959 and DE-A 24 47 825 (Mo₁₂BiFe₃Co_{4.5}Ni_{2.5}W_{0.5}K_{0.1}Oₓ).

Suitable multimetal oxides and their preparation are also described in U.S. Pat. No. 4,423,281 (Mo₁₂BiNi₈Pb0.5Cr₃K0.2Oₓ and Mo₁₂Bi_{b}Ni₇Al₃Cr_{0.5}K_{0.5}Oₓ), U.S. Pat. No. 4,336,409 (Mo₁₂BiNi₆Cd₂Cr₃P_{0.5}Oₓ), DE-A 26 00 128 (Mo₁₂BiNi_{0.5}Cr₃P_{0.5}Mg_{7.5}K_{0.1}OₓSiO₂) and DE-A 24 40 329 (Mo₁₂BiCo_{4.5}Ni_{2.5}Cr₃P_{0.5}K_{0.1}Oₓ).

Particularly preferred catalytically active multimetal oxides comprising molybdenum and at least one further metal have the general formula (la):

Mo₁₂BiₐFe_{b}Co_{c}Ni_{d}CrₑX¹_{f}X²_{g}O_{y} (Ia),

where
X¹=Si, Mn and/or Al,
X²=Li, Na, K, Cs and/or Rb,
0.2≦a≦1,
0.5≦b≦10,
0≦c≦10,
0≦d≦10,
2≦c+d≦10
0≦e≦2,
0≦f≦10
0≦g≦0.5
y=a number which is determined by the valence an abundance of the elements other than oxygen in (Ia) in order to achieve charge neutrality.

Preference is given to catalysts whose catalytically active oxide composition comprises only Co from among the two metals Co and Ni (d=0). X¹ is preferably Si and/or Mn and X² is preferably K, Na and/or Cs, with particular preference being given to X²=K.

The stoichiometric coefficient a in formula (Ia) is preferably such that 0.4≦a≦1, particularly preferably 0.4≦a≦0.95. The value of the variable b is preferably in the range 1≦b≦5 and particularly preferably in the range 2≦b≦4. The sum of the stoichiometric coefficient c+d is preferably in the range 4≦c+d≦8 and particularly preferably in the range 6≦c+d≦8. The stoichiometric coefficient e is preferably in the range 0.1≦e≦2 and particularly preferably in the range 0.2≦e≦1. The stoichiometric coefficient g is advantageously ≧0. Preference is given to 0.01≦g≦0.5 and particular preference is given to 0.05≦b≦0.2.

The value of the stoichiometric coefficient for oxygen, y, is determined by the valence and abundance of the cations in order to achieve charge neutrality. Coated catalysts according to the invention having catalytically active oxide compositions whose molar ratio of Co/Ni is at least 2:1, preferably at least 3:1 and particularly preferably at least 4:1, are advantageous. It is best for only Co to be present.

The coated catalyst is produced by applying a layer comprising the multimetal oxide comprising molybdenum and at least one further metal to the support by means of a binder, drying and calcining the coated support.

Finely divided multimetal oxides comprising molybdenum and at least one further metal which are to be used according to the invention can in principle be obtained by producing an intimate dry mixture of starting compounds of the elemental constituents of the catalytically active oxide composition and thermally treating the intimate dry mixture at a temperature of from 150 to 650° C.

The above-described heterogeneous, particulate multimetal oxide catalyst can have been introduced into the catalyst tubes of the two or more shell-and-tube reactors in a single zone or in two or more zones.

These zones can consist of pure catalyst or have been diluted with a material which does not react with the feedstream or components of the product gas from the reaction. Furthermore, the catalyst zones can consist of all-active material and/or of supported coated catalysts.

For the feedstream comprising the n-butenes it is possible to use pure n-butenes (1-butene and/or cis-/trans-2-butene) or else a gas mixture comprising butenes. Such a gas mixture can be obtained, for example, by nonoxidative dehydrogenation of n-butane. It is also possible to use a fraction which comprises n-butenes (1-butene and/or 2-butene) as main constituent and has been obtained from the C₄ fraction from naphtha cracking by separating off 1,3-butadiene and isobutene. Furthermore, gas mixtures which comprise pure 1-butene, cis-2-butene, trans-2-butene or mixtures thereof and have been obtained by dimerization of ethylene can also be used as feedstream. In addition, gas mixtures which comprise n-butenes and have been obtained by fluid catalytic cracking (FCC) can be used as feedstream.

In an embodiment of the process of the invention, the feedstream comprising the n-butenes is obtained by nonoxidative dehydrogenation of n-butane. The coupling of a nonoxidative catalytic dehydrogenation with the oxidative dehydrogenation of the n-butenes formed makes it possible to obtain a high yield of 1,3-butadiene, based on n-butane used. The nonoxidative catalytic dehydrogenation of n-butane gives a gas mixture comprising secondary constituents in addition to 1,3-butadiene, 1-butene, 2-butene and unreacted n-butane. Usual secondary constituents are hydrogen, water vapor, nitrogen, CO and CO₂, methane, ethane, ethene, propane and propene. The composition of the gas mixture leaving the first dehydrogenation zone can vary greatly depending on the mode of operation of the dehydrogenation. Thus, when the dehydrogenation is carried out with introduction of oxygen and additional hydrogen, the product gas mixture has a comparatively high content of water vapor and carbon oxides. In the case of a mode of operation without introduction of oxygen, the product gas mixture from the nonoxidative dehydrogenation has a comparatively high content of hydrogen.

The product gas stream from the nonoxidative dehydrogenation of n-butane typically comprises from 0.1 to 15% by volume of 1,3-butadiene, from 1 to 15% by volume of 1-butene, from 1 to 25% by volume of 2-butene (cis/trans-2-butene), from 20 to 70% by volume of n-butane, from 1 to 70% by volume of water vapor, from 0 to 10% by volume of low-boiling hydrocarbons (methane, ethane, ethene, propane and propene), from 0.1 to 40% by volume of hydrogen, from 0 to 70% by volume of nitrogen and from 0 to 5% by volume of carbon oxides. The product gas stream from the nonoxidative dehydrogenation can be fed without further work-up to the oxidative dehydrogenation.

Furthermore, any impurities in a range in which the effectiveness of the present invention is not inhibited can be present in the feedstream to the oxydehydrogenation. In the preparation of 1,3-butadiene from n-butenes (1-butene and cis/trans-2-butene),. saturated and unsaturated, branched and unbranched hydrocarbons such as methane, ethane, ethene, acetylene, propane, propene, propyne, n-butane, isobutane, isobutene, n-pentane and also dienes such as 1,2-or 1,3-butadiene may be mentioned as impurities. The amounts of impurities are generally 70% or less, preferably 50% or less, more preferably 40% or less and particularly preferably 30% or less. The concentration of linear monoolefins having 4 or more carbon atoms (n-butenes and higher homologues) in the feedstream is not subject to any particular restrictions; it is generally 35.00-99.99% by volume, preferably 50.00-99.0% by volume and even more preferably 60.00-95.0% by volume.

To carry out the oxidative dehydrogenation with full conversion of butenes, a gas mixture having a molar oxygen: n-butene ratio of at least 0.5 is required. Preference is given to working at an oxygen: n-butene ratio of from 0.55 to 10. To set this value, the starting material gas can be mixed with oxygen or an oxygen-comprising gas, for example air, and optionally additional inert gas or steam. The oxygen-comprising gas mixture obtained is then fed to the oxydehydrogenation.

The gas stream comprising molecular oxygen which is used according to the invention is a gas which generally comprises more than 10% by volume, preferably more than 15% by volume and more preferably more than 20% by volume, of molecular oxygen and is specifically preferably air, The upper limit to the content of molecular oxygen is generally 50% by volume or less, preferably 30% by volume or less and even more preferably 25% by volume or less. In addition, any inert gases in a range in which the effectiveness of the present invention is not inhibited can be present in the gas comprising molecular oxygen. As possible inert gases, mention may be made of nitrogen, argon, neon, helium, CO, CO₂ and water, The amount of inert gases is in the case of nitrogen generally 90% by volume or less, preferably 85% by volume or less and more preferably 80% by volume or less. In the case of constituents other than nitrogen, the amount is generally 20% by volume or less, preferably 10% by volume or less, If this amount becomes too large, it becomes increasingly more difficult to supply the reaction with the necessary oxygen.

Furthermore, inert gases such as nitrogen and also water (as water vapor) can also be fed in together with the mixed gas as feedstream and the gas stream comprising molecular oxygen. Nitrogen serves to set the oxygen concentration and to prevent formation of an explosive gas mixture, and the same applies to water vapor. Water vapor also serves to control carbonization of the catalyst and to remove the heat of reaction. Preference is given to mixing water (as water vapor) and nitrogen into the mixed gas and introducing this into the reactor. When water vapor is introduced into the reactor, it is preferably introduced in a proportion of 0.01-5.0 (parts by volume), preferably 0.1-3 and even more preferably 0.2-2.0, based on the amount of the abovementioned feedstream introduced. When nitrogen is introduced into the reactor, it is preferably introduced in a proportion of 0.1-8.0 (parts by volume), preferably 0.5-5.0 and even more preferably 0.8-3.0, based on the amount of the abovementioned feedstream introduced.

The proportion of the hydrocarbon-comprising feedstream in the mixed gas is generally 4.0% by volume or more, preferably 5.0% by volume or more and even more preferably 6.0% by volume or more. On the other hand, the upper limit is 20% by volume or less, preferably 15.0% by volume or less and even more preferably 12.0% by volume or less. To reliably avoid the formation of explosive gas mixtures, nitrogen gas is firstly introduced into the feedstream or into the gas comprising molecular oxygen before the mixed gas is obtained, the feedstream and the gas comprising molecular oxygen are mixed so as to give the mixed gas and this mixed gas is then preferably introduced.

During stable operation, the residence time in the production mode in the present invention is not subject to any particular restrictions, but the lower limit is generally 0.3 s or more, preferably 0.7 s or more and even more preferably 1.0 s or more. The upper limit is 5.0 s or less, preferably 3.5 s or less and even more preferably 2.5 s or less. The ratio of flow of mixed gas to the amount of catalyst in the interior of the reactor is 500-8000 h⁻¹, preferably 800-4000 h⁻¹ and even more preferably 1200-3500 h⁻¹. The butene load over the catalyst (expressed in g_{butenes}/(g_{catalyst}*hour) in stable operation is generally 0.1-5.0 h⁻¹, preferably 0.2-3.0 h⁻¹, and even more preferably 0.25-1.0 h⁻¹. Volume and mass of the catalyst relate to the complete catalyst consisting of support and active composition, insofar as a coated catalyst is used.

### Regeneration Mode

According to the invention, the process comprises a production mode and a regeneration mode which are operated alternately. In particular, each of the two or more shell-and-tube reactors is operated alternately in the production mode and the regeneration mode. Here, the switching-over from the production mode to the regeneration mode is generally carried out when the relative decrease in conversion (i.e. based on the conversion at the beginning of the respective production mode) at a constant temperature is not more than 25%. Operation is preferably switched over to the regeneration mode before the relative decrease in conversion at constant temperature is greater than 15%, in particular before the relative decrease in conversion at constant temperature is greater than 10%. In general, a regeneration mode is carried out only when the relative decrease in conversion at constant temperature is at least 2%.

In general, the production mode has a duration of from 5 to 5000 hours until a relative decrease in conversion of up to 25%, based on the conversion at the beginning of the production mode, is reached. The catalyst can go through up to 5000 or more cycles of production mode and regeneration mode.

The reaction temperature of the oxydehydrogenation in the regeneration mode is regulated by means of a heat transfer medium which circulates in the intermediate space around the catalyst tubes. The temperature of the heat transfer medium in the intermediate space between the catalyst tubes of the two or more shell-and-tube reactors corresponds to the temperature in the production mode and is preferably maintained at a value in the range from 330 to 450° C., preferably at a value in the range from 360 to 390° C., particularly preferably at a value in the range from 370 to 385° C. In general, two successive production modes are operated at essentially the same temperature (i.e. within a temperature window of ±2° C.). All temperatures mentioned above and below for production mode and regeneration mode relate to the temperature of the heat transfer medium in the inlet region of the heat transfer medium into the reactor.

The inlet region can be a ring channel in which the heat transfer medium flows via openings in the reactor wall into the space within the shell, or a chamber in the case of a twin reactor. Measuring elements for measuring the temperature are in each case arranged in the inlet region. These enable the temperatures specified to be set.

The activity of the multimetal oxide catalyst after each regeneration mode is generally restored to more than 95%, preferably more than 98% and in particular more than 99%, based on the activity of the multimetal oxide catalyst at the beginning of the preceding production mode.

One regeneration mode is operated between each two production modes. Operation is generally switched over to the regeneration mode before the decrease in conversion at constant temperature is greater than 25%. The regeneration mode is carried out by passing an oxygen-comprising regeneration gas mixture over the fixed catalyst bed at a temperature of from 350 to 490° C., as a result of which the carbon deposited on the multimetal oxide catalyst is burnt off.

The regeneration mode preferably comprises the following steps:
flushing the catalyst tubes comprising the multimetal oxide catalyst with inert gas, in particular nitrogen, and
treating the multimetal oxide catalyst comprised in the catalyst tubes with an oxygen-comprising regeneration gas.

The shell-and-tube reactor is flushed a number of times with inert gas until from two to five times the reactor volume has been replaced. The inert gas is in each case discharged. At the end of flushing with inert gas, the inert gas is also circulated via a compressor.

The flushing phase of the regeneration mode is followed by the actual regeneration phase in which an oxygen-comprising regeneration gas, in particular air, particularly preferably lean air, is introduced into the inert gas stream and circulated through the shell-and-tube reactor and a compressor. A heat exchanger is advantageously arranged upstream of the compressor. A substream of the oxygen-comprising regeneration gas is discharged upstream of the compressor.

The oxygen-comprising regeneration gas mixture used in the regeneration mode generally comprises an oxygen-comprising gas and additional inert gases, water vapor and/or hydrocarbons. In general, it comprises from 0.1 to 22% by volume, preferably from 0.1 to 10% by volume and in particular from 1 to 5% by volume, of oxygen.

A preferred oxygen-comprising gas which is present in the regeneration gas mixture is air. To produce the oxygen-comprising regeneration gas mixture, inert gases, water vapor and/or hydrocarbons can optionally be additionally mixed into the oxygen-comprising gas. As possible inert gases, mention may be made of nitrogen, argon, neon, helium, CO and CO₂. The amount of inert gases is, in the case of nitrogen, generally 99% by volume or less, preferably 98% by volume or less and even more preferably 97% by volume or less. In the case of constituents other than nitrogen, it is generally 30% by volume or less, preferably 20% by volume or less. The amount of oxygen-comprising gas is selected so that the proportion by volume of molecular oxygen in the regeneration gas mixture at the commencement of regeneration is 0 to 22%, preferably 0.5 to 10% and even more preferably 1 to 5%. The proportion of molecular oxygen can be increased during the course of the regeneration.

Furthermore, water vapor can also be comprised in the oxygen-comprising regeneration gas mixture. Nitrogen serves to adjust the oxygen concentration, and the same applies to water vapor. Water vapor can also be present in order to remove the heat of reaction and as mild oxidant for the removal of carbon-comprising deposits. Water (as water vapor) and nitrogen are preferably mixed into the regeneration gas mixture and introduced into the reactor. When water vapor is introduced into the reactor at the beginning of the regeneration, preference is given to introducing a proportion by volume of 0 to 50%, preferably 0.5 to 22% and even more preferably 1 to 10%. The proportion of water vapor can be increased during the course of the regeneration. The amount of nitrogen is selected so that the proportion by volume of molecular nitrogen in the regeneration gas mixture at the beginning of the regeneration is 20 to 99%, preferably 50 to 98% and even more preferably 70 to 97%. The proportion of nitrogen can be reduced during the course of the regeneration.

Furthermore, the regeneration gas mixture can comprise hydrocarbons. These can be added in addition to or instead of the inert gases. The proportion by volume of the hydrocarbons in the oxygen-comprising regeneration gas mixture is generally less than 50%, preferably less than 30% and more preferably less than 10%. The hydrocarbons can comprise saturated and unsaturated, branched and unbranched hydrocarbons such as methane, ethane, ethene, acetylene, propane, propene, propyne, n-butane, isobutane, n-butene, isobutene, n-pentane and also dienes such as 1,3-butadiene and 1,2-butadiene. In particular, they comprise hydrocarbons which do not display any reactivity in the presence of oxygen under the regeneration conditions in the presence of the catalyst.

During stable operation, the residence time in the regeneration mode during regeneration according to the present invention is not subject to any particular restrictions, but the lower limit is generally 0.3 s or more, preferably 0.7 s or more and even more preferably 1.0 s or more. The ratio of throughput of mixed gas to the catalyst volume in the interior of the reactor is 1 to 8000 h⁻¹, preferably 2 to 4000 h⁻¹ and even more preferably 5 to 3500 h⁻¹.

The regeneration mode is preferably carried out at essentially the same pressures as the production mode. In general, the reactor inlet pressure is <3 bar (gauge), preferably <2 bar (gauge) and particularly preferably <1.5 bar (gauge). In general, the reactor outlet pressure is <2.8 bar (gauge), preferably <1.8 bar (gauge) and particularly preferably <1.3 bar (gauge). A reactor inlet pressure which is sufficient to overcome flow resistances present in the plant and the downstream work-up is selected. In general, the reactor inlet pressure is at least 0.01 bar (gauge), preferably at least 0.1 bar (gauge) and particularly preferably 0.5 bar (gauge). In general, the reactor outlet pressure is at least 0.01 bar (gauge), preferably at least 0.1 bar (gauge) and particularly preferably 0.2 bar (gauge). The pressure drop over the entire catalyst bed is generally from 0.01 to 2 bar (gauge), preferably from 0.1 to 1.5 bar, particularly preferably from 0.4 to 1.0 bar.

The reaction temperature in the regeneration is controlled by means of a heat transfer medium which circulates in the intermediate space around the catalyst tubes. Possible liquid heat transfer media of this type are, for example, melts of salts such as potassium nitrate, potassium nitrite, sodium nitrite and/or sodium nitrate and also melts of metals such as sodium, mercury and alloys of various metals. However, ionic liquids or heat transfer oils can also be used. The temperature of the heat transfer medium is in the range from 330 to 490° C. and preferably from 350 to 450° C. and particularly preferably from 365 to 420° C. The temperatures mentioned relate to the temperature of the heat transfer medium at the inlet for the heat transfer medium on the reactor.

The product gas stream leaving the oxidative dehydrogenation is passed to a work-up which can be carried out in any known way.

The above-described process is preferably carried out continuously.

The external cooler is preferably a salt bath cooler and the secondary heat transfer medium is preferably water which partly or completely vaporizes in the salt bath cooler.

In particular, at least one of the two or more shell-and-tube reactors is operated in the regeneration mode as soon as the above-described switch-over from the production mode to the regeneration mode is necessary in one of the two or more shell-and-tube reactors, and the heat of reaction liberated in the remainder of the two or more shell-and-tube reactors which continue to be operated in the production mode minus the quantity of heat consumed for heating the input stream to the reaction temperature in the production mode is partly removed via the external cooler and the remainder is used to keep the temperature of the heat transfer medium in the intermediate spaces between the catalyst tubes of all shell-and-tube reactors constant with a fluctuation range of not more than +/−10° C.

The input stream generally has a temperature which is below the reaction temperature in order to avoid premature reactions and the disadvantages associated therewith. The reaction temperature should in general be reached only when the input stream comes into contact with the heterogeneous particulate catalyst.

The temperature of the heat transfer medium in the intermediate space between the tubes of all shell-and-tube reactors is more preferably kept constant with a fluctuation range of +/−5° C.

In a preferred embodiment, two shell-and-tube reactors are used.

In a further preferred embodiment, from 3 to 5 shell-and-tube reactors are used.

It is advantageous for all shell-and-tube reactors to have the same capacity in respect of 1,3-butadiene.

The capacity in respect of 1,3-butadiene of the two or more shell-and-tube reactors more preferably differs by ±10 to ±30%.

In particular, the catalyst tubes of the two or more shell-and-tube reactors have an internal diameter in the range from 15 to 50 mm, preferably from 20 to 35 mm.

The invention also provides a plant for carrying out the above-described process comprising two shell-and-tube reactors which each have a plurality of catalyst tubes into which a heterogeneous particulate multimetal oxide catalyst comprising molybdenum and at least one further metal as active composition has been introduced and also in each case comprising an upper ring line and a lower ring line at the upper and lower end, respectively, of each shell-and-tube reactor, which is connected to the intermediate spaces between the catalyst tubes and in which a heat transfer medium is circulated by means of a pump in each case, where the lower ring line of each of the shell-and-tube reactors is connected to the upper ring line of the other shell-and-tube reactor via a connecting line which can be closed or partly or fully opened by means of a shutoff device and also comprising an open equalization line which is physically separate from the connecting lines and connects the upper ring lines, and comprising an external cooler which is connected to each of the lower ring lines in each case via an input line which can be regulated by means of a slide valve in each case and is connected to each of the upper ring lines by means of a discharge line in each case.

A further preferred embodiment provides a compact plant, which can be referred to as twin reactor, comprising two shell-and-tube reactors having parallel longitudinal axes, having in each case a plurality of catalyst tubes into which a heterogeneous particulate multimetal oxide catalyst comprising molybdenum and at least one further metal as active composition has been introduced,

comprising an intermediate chamber between the two shell-and-tube reactors, which is open to the intermediate spaces between the catalyst tubes of the shell-and-tube reactors as a result of openings being provided in the mutually opposite subregions of the reactor shell of the shell-and-tube reactors and which is closed toward the outside by means of two longitudinal walls and an upper cover and a lower cover,
comprising three or more deflection plates which are alternately configured as disk-shaped deflection plates which extend over the cross section of both reactors and the intermediate chamber and leave passages free in the outer regions facing away from one another of the two reactors and as deflection plates which extend completely through the cross section of each reactor but leave the region of the intermediate chamber open,
where the shell-and-tube reactors are free of catalyst tubes in the deflection regions of the deflection plates
and the intermediate chamber is connected to an external cooler
and a heat transfer medium is conveyed by means of a pump through the intermediate space between the catalyst tubes of the shell-and-tube reactors.

The invention is illustrated in more detail below with the aid of a drawing and examples.

### The individual figures show:

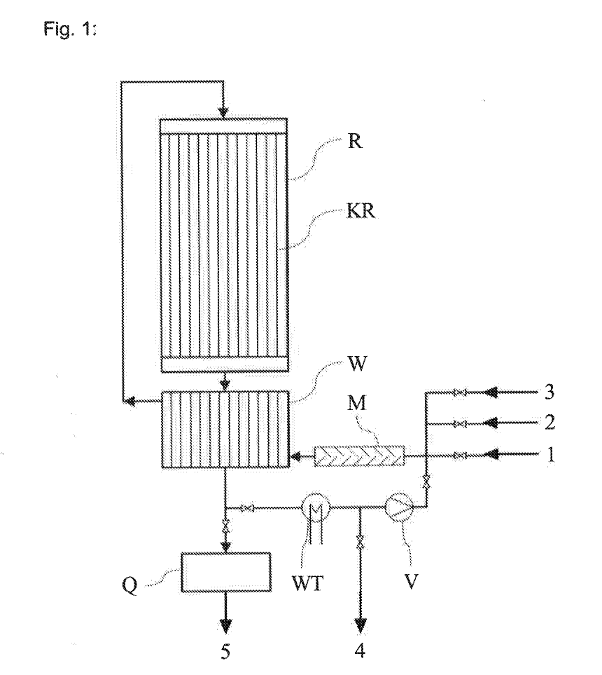

FIG. 1 a process layout according to the prior art (1-reactor design);

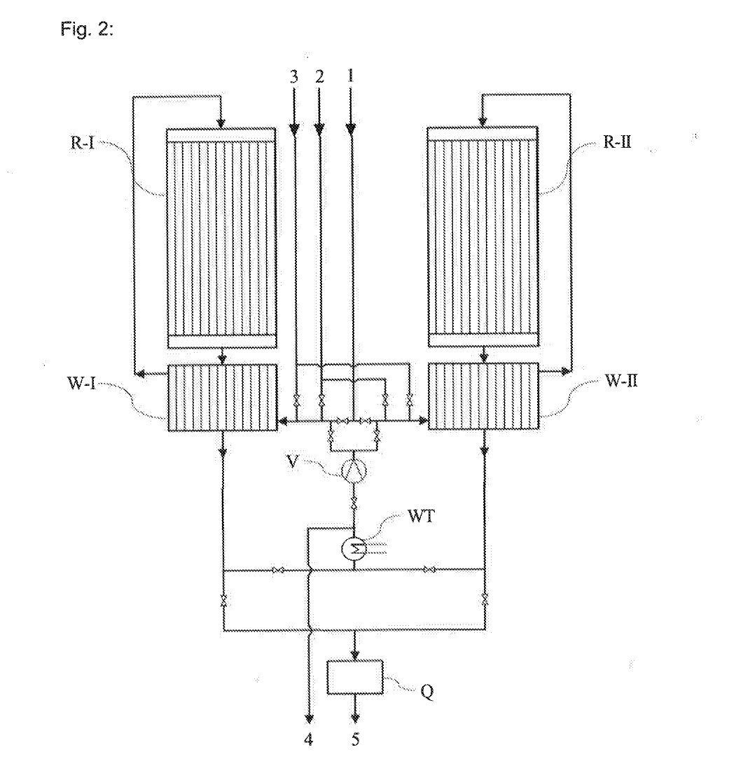

FIG. 2 a preferred process layout according to the invention (2-reactor design), where FIG. 2 depicts only the plant components relevant for conveying the gas streams both in the production mode and in the regeneration mode;

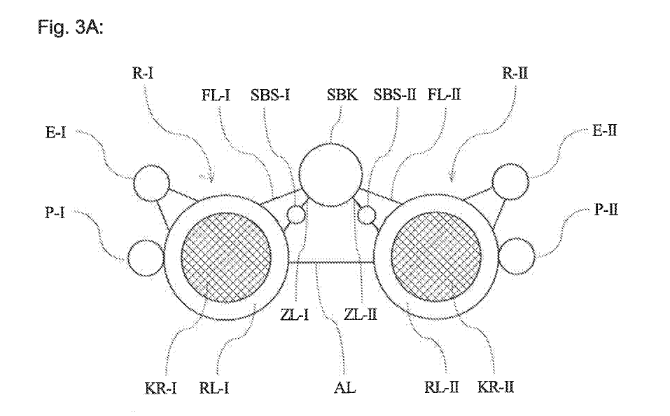

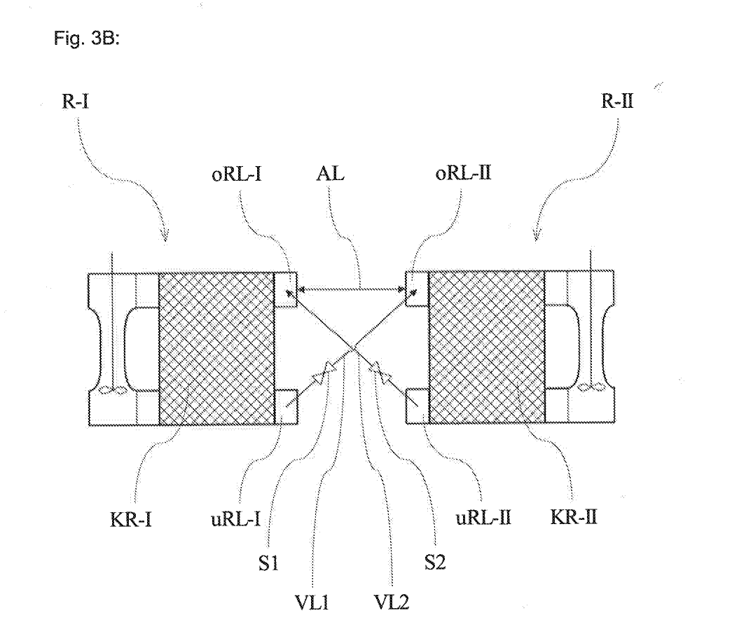

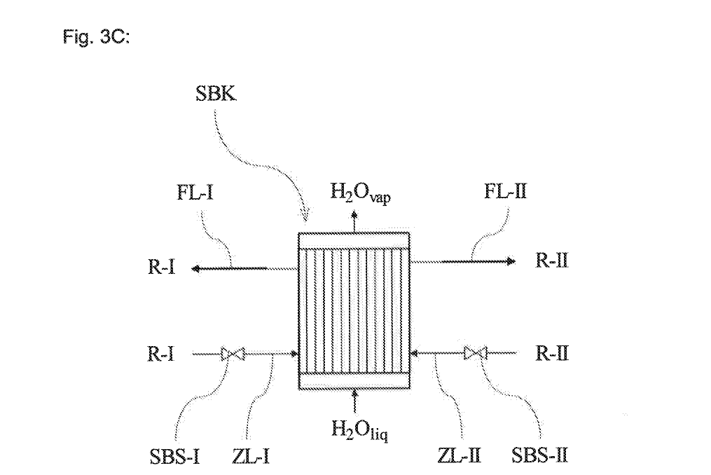

FIGS. 3A, 3B, 3C schematic depictions of a preferred process layout according to the invention (2-reactor design), where the plant components relevant for conveying the heat transfer medium are depicted;

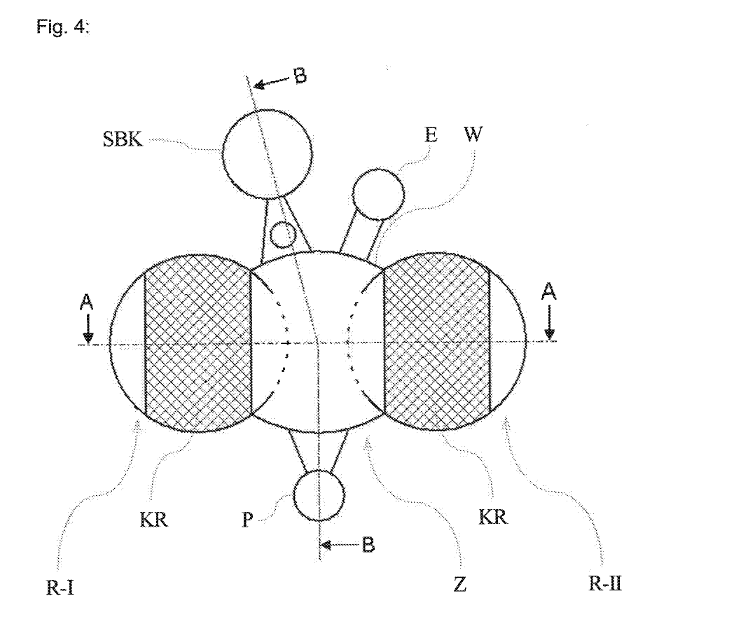

FIG. 4 a cross-sectional view through a particularly preferred, compact embodiment of a plant according to the invention (twin reactor), with this being depicted in

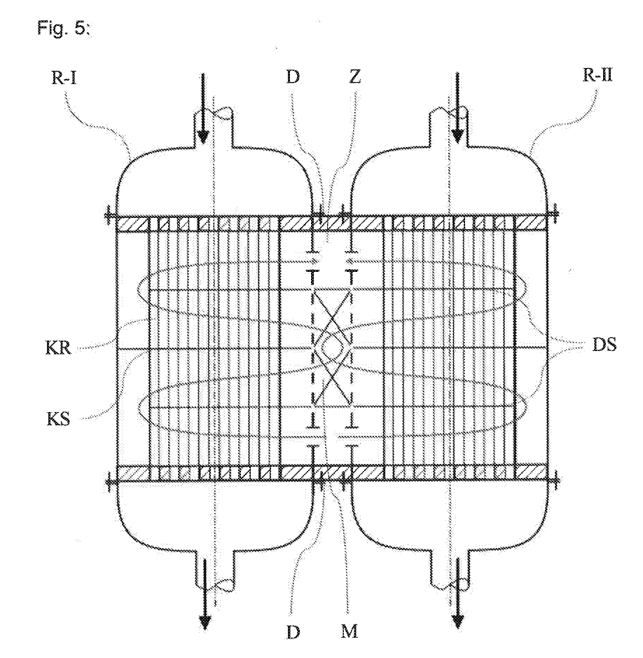

FIG. 5 along the section A-A and in

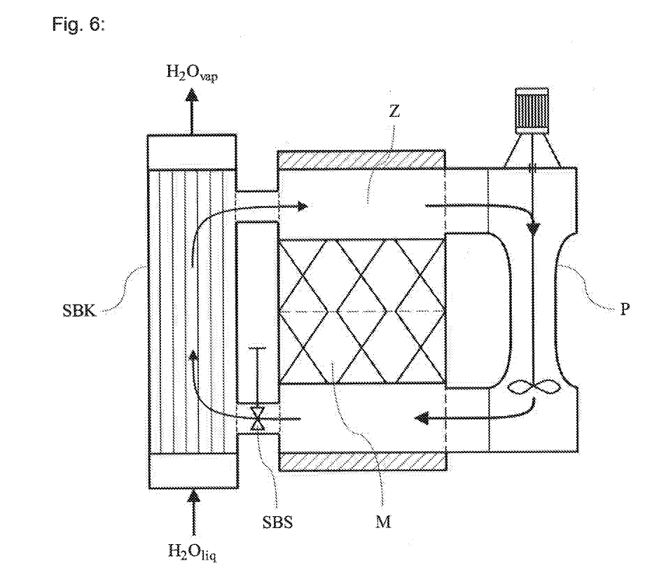

FIG. 6 along the section B-B;

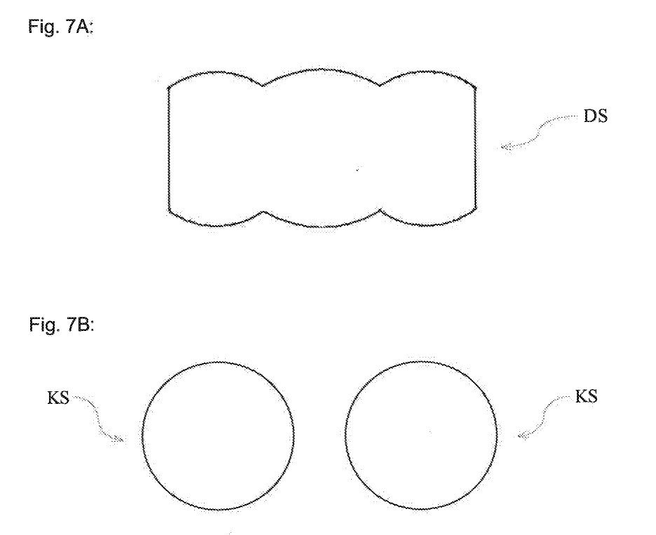

FIGS. 7A, 7B cross-sectional views through deflection plates DS which extend over the cross section of the two reactors and the intermediate chamber Z and leave passages free in the outer regions facing away from one another of the two reactors R-I, R-II or are configured as two disk-shaped deflection plates RS.

In the figures, identical reference symbols denote identical or corresponding components.

The schematic depiction in FIG. 1 shows, by way of example, a plant according to the prior art (1-reactor design), in which a gas-phase dehydrogenation and a regeneration of the exhausted catalyst can be carried out alternately in a single shell-and-tube reactor (R):

an input stream 1 obtained by mixing a feedstream comprising the n-butenes with an oxygen-comprising gas stream is conveyed through a static mixer M and, after preheating by means of the product gas mixture flowing out from the shell-and-tube reactor R in a cross-current heat exchanger W, fed into the shell-and-tube reactor R in the upper region of said reactor, flows through the catalyst tubes KR of said reactor, into which a heterogeneous, particulate multimetal oxide catalyst comprising molybdenum and at least one further metal as active composition has been introduced, resulting in the heterogeneously catalyzed oxidative dehydrogenation of n-butenes to 1,3-butadiene. The product gas mixture leaves the shell-and-tube reactor R at the lower end of said reactor and enters the cross-flow heat exchanger W in which it, as described, preheats the input stream to the shell-and-tube reactor R and is subsequently taken off via a quench Q (reaction mode).

For regeneration, the introduction of the stream 1 is interrupted and the reactor is flushed by introduction of inert gas, in particular nitrogen (stream 2): stream 2 is likewise conveyed via the static mixer M through the cross-current heat exchanger W and from the top downward through the catalyst tubes KR of the shell-and-tube reactor R and subsequently, not taken off by the quench Q like the product gas mixture, but instead discharged via line 4, with flushing being carried out a number of times until from 3 to 5 times the reactor volume has been replaced. At the end of the flushing phase, stream 2 can also be circulated via the additional heat exchanger WT and compressor V.

The flushing phase of the regeneration mode is followed by the actual regeneration phase in which the introduction of the inert gas stream 2 is interrupted and regeneration gas, in particular air, particularly preferably lean air, stream 3, is instead fed in. Stream 3 is likewise conveyed via the static mixer M through the cross-current heat exchanger W and from the top downward through the catalyst tubes KR of the shell-and-tube reactor R, but is subsequently circulated via an additional heat exchanger WT and a compressor V. Instead of the additional heat exchanger WT, it is also possible to use a further quench Q.

FIG. 2 shows, on the other hand, a schematic depiction of a preferred embodiment according to the invention (2-reactor design), with only the path of the gas streams, but not of the heat exchanger, being shown:

In the reaction mode, a stream 1 as described above which has been obtained by mixing a feedstream comprising the n-butenes with an oxygen-comprising gas stream and is preheated beforehand by the product gas mixture leaving the respective shell-and-tube reactor R-I, R-II by means of in each case one cross-flow heat exchanger (W) is fed into each of the two shell-and-tube reactors R-I, R-II in the upper region of the respective reactor. The product gas mixture flows from each of the shell-and-tube reactors R-I, R-II from the lower region of the respective reactor, heats the input stream in the cross-flow heat exchanger W and is subsequently cooled in a quench Q. In the preferred embodiment depicted in FIG. 2, the two streams exiting from the cross-flow heat exchangers W are combined before being fed to the quench Q. However, it is also possible, for example, for each reactor to be followed by a dedicated quench, etc.

For regeneration, the reactor concerned is switched over from the reaction mode to the regeneration mode, with the other reactor(s), in the present embodiment reactor R-I, continuing to be operated in the reaction mode. For this purpose, stream 1 is still fed into the reactor R-I but not into the reactor R-II which is instead firstly flushed with inert gas, in particular nitrogen, stream 2. Stream 2 is conveyed through the cross-current heat exchanger W and from the top downward through the catalyst tubes KR of the shell-and-tube reactor R and subsequently discharged via line 4, with flushing being carried out a number of times until from 3 to 5 times the reactor volume has been replaced. At the end of the flushing phase, stream 2 can also be circulated via the additional heat exchanger WT and the compressor V.

After the flushing phase is complete, the actual regeneration phase is started by interrupting the introduction of the stream 2 and instead circulating a stream 3 comprising air, in particular lean air, through the reactor RA:

the flushing phase of the regeneration mode is followed by the actual regeneration phase in which the introduction of the inert gas stream 2 is interrupted and regeneration gas, in particular air, particularly preferably lean air, stream 3, is instead fed in. Stream 3 is likewise conveyed via the cross-current heat exchanger W and from the top downward through the catalyst tubes KR of the shell-and-tube reactor R, but subsequently circulated via an additional heat exchanger WT and a compressor V. Instead of the additional heat exchanger WT, it is also possible to use a further quench Q.

FIGS. 3A to 3C, on the other hand, show the path of the heat transfer medium for the same inventive embodiment (2-reactor design) depicted in FIG. 2 for the path of the gas streams:

the cross-sectional view in FIG. 3A shows the two shell-and-tube reactors R-I, R-II, with schematically indicated sections through the catalyst tubes KR and also ring lines RL for the heat transfer medium. An electric heater E-I, E-II is provided for each of the two shell-and-tube reactors R-I, R-II. The heat transfer medium is in each case conveyed by means of a pump P-I, P-II. The ring lines RL are each connected via an input line ZL-I, ZL-II, which is regulated by means of salt bath slide valves SBS-I, SBS-II, and via discharge lines FL-I, FL-II to a salt bath cooler SBK. An equalization line AL is provided between the ring lines RL of the two shell-and-tube reactors R-I, R-II.

The longitudinal section depicted in FIG. 3B shows the connection of the lower ring line uRL-I of the shell-and-tube reactor R-I to the upper ring line oRL-II of the second shell-and-tube reactor R-II via a connecting line VL having a connecting slide valve S1 or of the lower ring line uRL-II of the second shell-and-tube reactor R-II to the upper ring line oRL-I of the first shell-and-tube reactor R-I via a connecting line VL having a connecting slide valve S2. P+ and p− denote the pressure and suction sides, respectively, for the flow of the heat exchange medium. The two upper ring lines oRL-I, oRL-II are connected via an open equalization line AL.

FIG. 3C schematically shows a longitudinal section through the salt bath cooler SBK, which is by way of example configured as a shell-and-tube heat exchanger, having input lines ZL-I, ZL-II regulated by means of salt bath slide valves SBS-I, SBS-II from the shell-and-tube reactors R-I, R-II and discharge lines FL-I, FL-II at the opposite end of the salt bath cooler SBK. As secondary heat transfer medium, use is made of, for example, water which forms steam in the salt bath cooler SBK.

FIG. 4 schematically shows a section through a particularly preferred, compact embodiment which can be referred to as twin reactor: the two shell-and-tube reactors R-I, R-II are connected to one another via an intermediate chamber Z which is closed to the outside by means of longitudinal walls W and covers A, which cannot be seen in the cross section depicted in FIG. 4, but communicates with the interior spaces of the two shell-and-tube reactors R-I, R-II via openings in the walls of said shell-and-tube reactors. A pump P, an electric heater E and an external cooler SBK are connected to the intermediate chamber Z. The cross-sectional depiction in FIG. 4 shows the advantageous embodiment in which the shell-and-tube reactors R-I, R-II are free of catalyst tubes KR in the regions where a change of direction occurs.

The longitudinal section in the plane A-A depicted in FIG. 5 also shows the covers A which close off the intermediate chamber Z at the upper and lower ends of said chamber, the mixer M arranged centrally in the intermediate chamber Z and, by way of example, two disk-shape deflection plates DS which are arranged alternately with two deflection disks KS in the transverse direction in the shell-and-tube reactors R-I, R-II. The arrows directed from the top downward in the two shell-and-tube reactors R-I. R-II indicate the flow direction for the gas (reaction gas mixture or regeneration gas) and the curved arrows in the interior space of the two shell-and-tube reactors R-I. R-II indicate the path of the heat transfer medium.

The section B-B depicted in FIG. 6 shows the arrangement of the external cooler SBK and of the pump P, the flow path of the heat transfer medium through the pump P, the intermediate chamber Z and the external cooler SBK. The static mixers M in the central region of the intermediate chamber Z and also the salt bath slide valve SBS can also clearly be seen in FIG. 6.

FIGS. 7A and 7B show cross-sectional views through deflection plates DS (in FIG. 7A) which extend over the cross section of the two reactors and the intermediate chamber Z and leave passages open in the outer regions facing away from one another of the two reactors R-I, R-II or are configured as two disk-shaped deflection plates RS (in FIG. 7B).

### EXAMPLES

### 1—Reactor Design (Alternate Dehydrogenation and Regeneration) (Prior Art) Regeneration Mode Using One Reactor

Salt bath reactor R

| | | |
|---|---|---|
| Regeneration temperature | 380° | C. |
| Inlet temperature | 300° | C. |
| Heat capacity | 1100 | J/kg/K |
| Volume flow per catalyst tube KR | 1500 | Standard l/h |
| Cat volume per catalyst tube KR | 2.5 | l |
| GHSV (gas hourly space velocity) | 600 | h⁻¹ |
| Number of catalyst tubes KR | 24000 | |
| Total volume flow (recycle gas) | 36000 | Standard m³/h |
| Mass flow (recycle gas) | 45 | t/h |
| Heat power (heating of recycle gas) | 1.1 | MW |
| Heat losses in reactor system | 0.5 | MW |
| Heat power to be introduced (salt bath heating by | 1.6 | MW |
| means of electric heaters) | | |

Cross-Current Heat Exchanger WW (Heating of the Regeneration Gas/Cooling of the Offgas Stream from the Reactor)

| Cold side | | |
|---|---|---|
| Inlet temperature of the lean air (stream 3) | 210° | C. |
| Outlet temperature | 300° | C. |
| Heat power taken up | 1.24 | MW |
| Hot side | | |
| Inlet temperature | 380° | C. |
| Outlet temperature | 290° | C. |
| Heat power released | 1.24 | MW |
| Cooling before compression by means | | |
| of additional heat exchanger | | |
| Temperature at compressor inlet | 210° | C. |
| Heat power of cooling | 1.10 | MW |

### Reaction Mode (Dehydrogenation Operation) Using One Reactor

Salt bath reactor R

| Butadiene capacity | 16 | t/h |
|---|---|---|
| | 0.082 | kmol/s |
| Butadiene yield | 80% | |
| Inlet concentration of n-butenes in stream 1 | 8% | |
| Inlet stream of n-butenes | 0.103 | kmol/s |
| Total volume —stream 1 | 1.29 | kmol/s |
| Total mass flow of reaction gas mixture | 134 | t/h |
| Volume flow per catalyst tube KR | 4.32 | Standard m³/h |
| GHSV (gas hourly space velocity) | 1728 | h⁻¹ |
| Reaction enthalpy | −135 | kJ/mol |
| Heat of reaction (oxydehydrogenation of n-butenes) | 11 | MW |
| Heat of reaction (total) | 22 | MW |
| Heating of stream 1 | 7.1 | MW |
| Reaction temperature (salt bath) | 380° | C. |
| Temperature of the product gas mixture at the | 382° | C. |
| reactor outlet | | |
| Heat to be removed (salt bath via salt bath cooler) | 14.9 | MW |

### Cross-Current Heat Exchanger WW (During the Reaction Mode)

| Cold side | | |
|---|---|---|
| Inlet temperature | 50° | C. |
| Outlet temperature | 210° | C. |
| Heat power taken up | 6.56 | MW |
| Hot side | | |
| Inlet temperature | 382° | C. |
| Outlet temperature | 222° | C. |
| Heat power released | 6.56 | MW |

### 2—Reactor Design (According to the Invention)

Reaction mode (dehydrogenation operation):

| Butadiene capacity | 16 | t/h |
|---|---|---|
| | 0.082 | kmol/s |
| Butadiene yield | 80% | |
| Inlet concentration of n-butenes in stream 1 | 8% | |
| Inlet stream of n-butenes | 0.103 | kmol/s |
| Total volume flow | 1.29 | kmol/s |
| Total mass flow | 134 | t/h |
| Volume flow per tube | 3.93 | Standard m³/h/tube |
| GHSV (gas hourly space velocity) | 1571 | h⁻¹ |
| Number of catalyst tubes KR (total) | 26400 | |
| Number of catalyst tubes KR per reactor R | 13200 | |

### 1 Reactor in the Reaction Mode and 1 Reactor in the Regeneration Mode

Salt bath reactor

| Load | 55% compared to | |
|---|---|---|
| | reactor in 1-reactor | |
| | design | |
| Reaction enthalpy | −135 | kJ/mol |
| Heat of reaction (oxydehydrogenation of n- | 6 | MW |
| butenes) | | |
| Heat of reaction (total) | 12 | MW |
| Heating of stream 1 | 3.9 | MW |
| Reaction temperature (salt bath) | 380° | C. |
| Temperature of the product gas mixture at the | 382° | C. |
| reactor outlet | | |
| Heat to be removed (by means of salt bath: salt | 7.1 | MW |
| bath cooler, salt bath system) | | |

### Cross-Current Heat Exchanger WW (During the Normal Reaction Mode)

| Cold side | | |
|---|---|---|
| Inet temperature | 50° | C. |
| Outlet temperature | 210° | C. |
| Heat power taken up | 3.61 | MW |
| Hot side | | |
| Inlet temperature | 382° | C. |
| Outlet temperature | 222° | C. |
| Heat power released | 3.61 | MW |

### Regeneration Phase: 1 Reactor in the Regeneration Mode

Salt bath reactor R

| | | |
|---|---|---|
| Regeneration temperature | 380° | C. |
| Inlet temperature | 250° | C. |
| Heat capacity | 1100 | J/kg/K |
| Volume flow per catalyst tube KR | 1500 | Standard l/h |
| Cat. volume per catalyst tube KR | 2.5 | L |
| GHSV (gas hourly space velocity) | 600 | h⁻¹ |
| Number of catalyst tubes KR | 13200 | |
| Total volume flow | 19800 | Standard m³/h |
| Mass flow | 24.75 | t/h |
| Heat power (heating of recycled gas) | 1.0 | MW |
| Heat losses in reactor system | 0.4 | MW |
| Heat power to be introduced (by means of salt | 1.4 | MW |
| bath system) | | |

Cross-Current Heat Exchanger WW Cheating of the Regeneration Gas/Cooling of the Offgas Stream from the Reactor)

| Cold side | | |
|---|---|---|
| Inlet temperature of the lean air | 90° | C. |
| Outlet temperature | 250° | C. |
| Heat power taken up | 1.21 | MW |
| Hot side | | |
| Inlet temperature | 380° | C. |
| Outlet temperature | 220° | C. |
| Heat power released | 1.21 | MW |
| Quench Q | | |
| Temperature | 50° | C. |
| Heat power (losses) | 1.3 | MW |

A salt bath reactor R which is particularly suitable for use in the oxydehydrogenation (reaction mode) and in the regeneration is described below by way of example:

| | | 1—Reactor | 2—Reactor |
|---|---|---|---|
| Reactor variant | Unit | design | design |
| Total butadiene | [t/h] | 16.0 | 16.0 |
| capacity | | | |
| Number of | [—] | 1 | 2 |
| reactor(s) R | | | |
| Butadiene capacity/ | [t/h] | 16.0 | 8.8 |
| reactor R | | | |
| Number of catalyst | [—] | 24000 | 13500 |
| tubes KR | | | |
| External diameter of | [mm] | 30.0 | 30.0 |
| tubes | | | |
| Wall thickness of tubes | [mm] | 2.0 | 2.0 |
| Tube separation | [mm] | 38.0 | 38.0 |
| Tube length | [mm] | 5000.0 | 5000.0 |
| Heat to be removed per | [MW] | 23.0 | 13.0 |
| reactor R | | | |
| Heat transfer medium | [—] | Salt melt | Salt melt |
| Average temperature | [° C.] | 380.00 | 380.0 |
| of heat transfer medium | | | |
| Density of heat transfer | [kg/m³] | 1800.7 | 1800.7 |
| medium | | | |
| Spec. heat capacity of | [J/(kg * K)] | 1534.4 | 1534.4 |
| heat transfer medium | | | |
| Temperature difference | [° C.] | 2.5 | 2.5 |
| (uRL − oRL) of salt | | | |
| melt | | | |
| Mass flow of salt melt | [t/h] | 21584.6 | 12200.0 |
| Volume flow of salt | [m³/h] | 11987.1 | 6775.3 |
| melt | | | |
| Number of salt pumps | | 2 | 1 |
| Area of internal change | [m²] | 3.03 | 1.71 |
| of direction (tube-free) | | | |
| Area occupied by tubes | [m²] | 30.01 | 16.88 |
| Area of external change | [m²] | 3.33 | 1.88 |
| of direction (tube-free) | | | |
| Wall thickness of | [mm] | 30.0 | 30.0 |
| cylindrical reactor wall | | | |
| Cross-sectional area of | [m²] | 0.55 | 0.31 |
| upper and lower ring | | | |
| channel | | | |
| Internal height of ring | [mm] | 800.0 | 800.0 |
| channel | | | |
| Internal width of ring | [mm] | 690.0 | 390.0 |
| channel | | | |
| Internal diameter of | [mm] | 1960.0 | 1480.0 |
| tube circle Ri | | | |
| External diameter of | [mm] | 6490.0 | 4870.0 |
| tube circle Ra | | | |
| Internal diameter of | [mm] | 6800.0 | 5110.0 |
| reactor wall | | | |
| External diameter of | [mm] | 6860.0 | 5170.0 |
| reactor | | | |
| External diameter of | [mm] | 8290.0 | 5990.0 |
| salt bath channel | | | |
| Reactor configuration | | | |
| Number of temperature- | [—] | 1 | 1 |
| control zones | | | |
| Flow path of the salt | [—] | radial (transverse | radial (transverse |
| melt | | flow against the | flow against the |
| | | tubes) | tubes) |
| min. Heat transfer | [W/(m² * K)] | 2000 | 2000 |
| coefficient on salt side | | | |
| Flow direction of the | [—] | Bottom upward | Bottom upward |
| salt melt | | | |
| Tubes in the deflection | [—] | None | None |
| regions | | | |
| Number of deflection | [—] | 4-6 | 4-6 |
| regions | | | |
| Type of deflection | [—] | Disk/rings | Disk/rings |
| plates | | (alternating) | (alternating) |
| Gap widths between | [mm] | 0.15-1.5 | 0.15-1.5 |
| catalyst tubes KR and | | | |
| deflection plates | | | |
| Thickness of deflection | [mm] | 10.0-20.0 | 10.0-20.0 |
| plates | | | |
| Additional holes in the | [—] | As required | As required |
| deflection plates | | | |

1-15. (canceled)

16. A process for preparing 1,3-butadiene by oxidative dehydrogenation of n-butenes over a heterogeneous particulate multimetal oxide catalyst which comprises molybdenum and at least one further metal as active composition and has been introduced into the catalyst tubes (KR) of two or more shell-and-tube reactors (R-I, R-II), where a heat transfer medium flows through the intermediate space between the catalyst tubes (KR) of the two or more shell-and-tube reactors (R-I, R-II),
and the process comprises a production mode and a regeneration mode which are operated alternately,
in the production mode, a feedstream comprising the n-butenes is mixed with an oxygen-comprising gas stream and passed as input stream (1) over the heterogeneous particulate multimetal oxide catalyst which has been introduced into the catalyst tubes (KR) of the two or more shell-and-tube reactors (R-I, R-II) and the heat transfer medium takes up, by indirect heat exchange, the heat of reaction liberated minus the quantity of heat which is consumed for heating the input stream (1) to the reaction temperature in the production mode and passes all or part of it onto a secondary heat transfer medium (H₂O_{liq}) in an external cooler (SPK) and, in the regeneration mode, the heterogeneous particulate multimetal oxide catalyst is regenerated by passing an oxygen-comprising gas mixture (3) over the catalyst and burning off the deposits which have deposited on the heterogeneous particulate multimetal oxide catalyst, wherein
the two or more shell-and-tube reactors (R-I, R-II) have a single heat transfer medium circuit and
the number of the two or more shell-and-tube reactors (R-I, R-II) which are operated in the production mode is always such that the heat of reaction liberated minus the quantity of heat consumed for heating the input stream (1) to the reaction temperature in the production mode is sufficient to keep the temperature of the heat transfer medium in the intermediate spaces between the catalyst tubes (KR) of all shell-and-tube reactors (R-I, R-II) constant with a fluctuation range of not more than +/−10° C.

17. The process according to claim 16, wherein the process is carried out continuously.

18. The process according to claim 16, wherein the heat transfer medium is a salt melt, the external cooler (SBK) is a salt bath cooler and the secondary heat transfer medium (H₂O_{liq}) is water which partly or completely evaporates in the salt bath cooler (SBK).

19. The process according to claim 16, wherein at least one of the two or more shell-and-tube reactors (R-I, R-II) is operated in the regeneration mode and the heat of reaction liberated in the others of the two or more shell-and-tube reactors (RI, R-II) which are operated in the production mode minus the quantity of heat which is consumed for heating the input stream (1) to the reaction temperature in the production mode is partly removed via the external cooler (SBK) and the remainder is utilized to keep the temperature of the heat transfer medium in the intermediate spaces between the catalyst tubes (KR) of all shell-and-tube reactors (RI, R-II) constant with a fluctuation range of not more than +/−10° C.

20. The process according to claim 16, wherein the heterogeneous particulate multimetal oxide catalyst is a coated catalyst formed by catalyst particles of a ceramic support which is enveloped by a shell comprising the active composition.

21. The process according to claim 16, wherein the temperature of the heat transfer medium in the intermediate space between the tubes of all shell-and-tube reactors (R-I, R-II) is kept constant with a fluctuation range of +/−5° C.

22. The process according to claim 16, wherein two shell-and-tube reactors (R-I, R-II) are used.

23. The process according to claim 16, wherein from 3 to 5 shell-and-tube reactors (R-I, R-II) are used.

24. The process according to claim 16, wherein all shell-and-tube reactors (R-I, R-II) have the same capacity in respect of 1,3-butadiene.

25. The process according to claim 16, wherein the capacity in respect of 1,3-butadiene of the two or more shell-and-tube reactors (R-I, R-II) differs by ±10 to ±30%.

26. The process according to claim 16, wherein the catalyst tubes (KR) of the two or more shell-and-tube reactors (R-I, R-II) have an internal diameter in the range from 15 to 50 mm.

27. The process according to claim 16, wherein the catalyst tubes (KR) of the two or more shell-and-tube reactors (R-I, R-II) have an internal diameter in the range from 20 to 35 mm.

28. The process according to claim 16, wherein the regeneration mode has the following regeneration steps:
flushing the catalyst tubes comprising the multimetal oxide catalyst with inert gas (2), and treating the multimetal oxide catalyst comprised in the catalyst tubes with an oxygen-comprising regeneration gas (3).

29. The process according to claim 28, wherein the inert gas (2) is nitrogen.

30. The process according to claim 16, wherein the temperature of the heat transfer medium in the intermediate space between the catalyst tubes (KR) of the two or more shell-and-tube reactors (R-I, R-II) is maintained at a value in the range from 350 to 420° C.

31. The process according to claim 16, wherein the temperature of the heat transfer medium in the intermediate space between the catalyst tubes (KR) of the two or more shell-and-tube reactors (R-I, R-II) is maintained at a value in the range from 370 to 385° C.

32. A plant for carrying out the process according to claim 22, comprising two shell-and-tube reactors (R-I, R-II) which each have a plurality of catalyst tubes (KR) into which a heterogeneous particulate multimetal oxide catalyst comprising molybdenum and at least one further metal as active composition has been introduced and also in each case comprising an upper ring line (oRL-I, oRL-II) and a lower ring line (uRL-uRL-II) at the upper and lower end, respectively, of each shell-and-tube reactor (R-I, R-II), which is connected to the intermediate spaces between the catalyst tubes (KR) and in which a heat transfer medium is circulated by means of a pump (P) in each case, where the lower ring line (uRL-I, uRL-II) of each of the shell-and-tube reactors (R-I, R-II) is connected to the upper ring line (oRL-I, oRL-II) of the other shell-and-tube reactor (R-I, R-II) via a connecting line (VL) which can be closed or partly or fully opened in each case by means of a shutoff device (S1, S2) and an open equalization line (AL) which is physically separate from the connecting lines (VL) connects the upper ring lines (oRL-I, oRL-II),
and comprising an external cooler (SBK) which is connected to each of the lower ring lines (uRL-I, uRL-II) in each case via an input line (ZL-I, ZL-II) which can be regulated by means of a slide valve (SBS-I, SBS-II) in each case and is connected to each of the upper ring lines (oRL-I, oRL-II) by means of a discharge line (FL-I, FL-II) in each case.

33. A plant for carrying out the process according to claim 22, comprising two shell-and-tube reactors (R-I, R-II) having parallel longitudinal axes, having in each case a plurality of catalyst tubes (KR) into which a heterogeneous particulate multimetal oxide catalyst comprising molybdenum and at least one further metal as active composition has been introduced,
comprising an intermediate chamber (Z) between the two shell-and-tube reactors (R-I, R-II), which is open to the intermediate spaces between the catalyst tubes (KR) of the shell-and-tube reactors (R-I, R-II) as a result of openings being provided in the mutually opposite subregions of the reactor shell of the shell-and-tube reactors (R-I-R-II) and
which is closed toward the outside by means of two longitudinal walls (W) and an upper cover and a lower cover (D),
comprising three or more deflection plates which are alternately configured as deflection plates (DS) which extend over the cross section of both reactors and the intermediate chamber (Z) and leave passages free in the outer regions facing away from one another of the two reactors (R-I, R-II) and as two disk-shaped deflection plates (RS) which extend completely through the cross section of each reactor (R-I, R-II) but leave the region of the intermediate chamber (Z) open, where the shell-and-tube reactors (R-I, R-II) are free of catalyst tubes (KR) in the deflection regions of the deflection plates (DS)
and the intermediate chamber (Z) is connected to an external cooler (SBK) and a heat transfer medium is conveyed by means of a pump (P) through the intermediate space between the catalyst tubes (KR) of the shell-and-tube reactors
(R-I, R-II), through the intermediate chamber (Z) and through the external cooler (SBK).
